(19) United States
(12) Patent Application Publication
(10) Pub. No.: US 2008/0275375 A1
(43) Pub. Date: Nov. 6, 2008

(54) ADHESIVE COMPOSITION FOR A HUMID MEDIUM, BASED ON BLOCK COPOLYMERS COMPRISING AT LEAST ONE HYDROPHILIC BLOCK

(76) Inventors: Olvier GUEREET, Domicile (FR); Nour Eddine El Bounia, Orthez (FR); Sylwia Poivet, Bordeaux (FR); Frederic Nallet, Bordeaux (FR); Pascale Fabre, Bordeaux (FR); Eric Papon, Saint-Magne-De-Castillon (FR); Yves Gnanou, Talence (FR); Kai Schierholz, Bordeaux (FR)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 12/171,993

(22) Filed: Jul. 11, 2008

Related U.S. Application Data
(63) Continuation of application No. 10/522,238, filed on Jan. 25, 2005.

(51) Int. Cl. C08F 267/10; A61F 13/02

(52) U.S. Cl. 602/41; 525/55

(57) ABSTRACT

The invention relates to a block copolymer which is produced by a controlled radical polymerization, has an individual composition and structure and is used for adhesion in a humid medium The inventive copolymer comprises a continuous flexible and water repellent matrix (of elastomer nature) and a dispersed phase in the form of hydrophilic nanodomains. Said copolymer exhibits an excellent adhesion as a result of the structure thereof also in a very humid medium.

The present invention relates to the field of block copolymers, in particular to the field of block copolymers containing at least one hydrophilic block, and more particularly to the use of such copolymers in adhesive formulations, especially adhesive formulations for use in a dry as well as in a moist medium.

The adhesive formulations of the invention can be used in a moist medium, particularly as regards any adhesion to the skin for a number of medical applications, (patches, prostheses) or paramedical applications (dressings) which have to stick even after passage through water, as well as to the preparation of labels.

Current solutions in the field of adhesion in a moist medium are of the acrylic gum (hot melt or latex), vinyl polyacetate, polyvinylpyrrolidone, silicone or polyurethane type. The majority of such products require chemical crosslinking of the hydrogen bond curing, UV curing, peroxide curing or wet curing type in the case of silanes. It should be noted that one of the key parameters in proposing an adhesive formulation intended for cutaneous application is the fact that solvents which are not biocompatible cannot be used; all solutions involved in creating an adhesive are produced either by hot melt or by in vivo reaction.

A further solution which can be envisaged to avoid using a solvent consists of using block copolymers the structure of which gives them the characteristics of crosslinked materials at the service temperature. In general, block copolymers used in the field of adhesive formulations are of the styrene/diene type. However, styrenic and dienic monomers are hydrophobic and when they are used to bond substrates in a moist medium, the water prevents good contact of the substrate surfaces with the adhesive. Wholly acrylic copolymers have also been envisaged (Mancinelli P. A., Seminar Proceeding (Pressure Sensitive Tape Council), 2-5 May 1989, 161-181), but the hydrophobic nature of acrylates and methacrylates does not allow adhesion in a moist medium.

Thus, the invention seeks to overcome the problem of developing formulae for block copolymers that can be used in adhesive formulations which bond both in a dry medium and in a moist medium.

The Applicant has discovered that the solution to the problems mentioned above resides in the use in the adhesive formulations of a block copolymer having at least one strongly hydrophilic block and at least one hydrophobic or very slightly hydrophilic block.

The term “very slightly hydrophilic” as used by the Applicant means blocks with a water absorption capacity we, as defined below, of less than 20% and preferably less than 10%.

The hydrophilic block is rigid and constitutes the minor phase dispersed in nanodomains (capsules, cylinders or lamellae), while the hydrophobic block is elastomeric in nature and constitutes the continuous phase.

The Applicant initially established that, for a block copolymer type adhesive, the continuous phase which allows adhesion is generally a hydrophobic phase as no truly hydrophilic and elastomeric monomers exist (with the exception of methoxyethyl acrylate, which the Applicant excluded from the investigations because of its toxicity). For this reason, moist medium adhesion is rendered difficult. The Applicant has surprisingly realized that hydrophilic polymer domains nanodispersed in the hydrophobic matrix can make the material bond even in a moist medium.

In a nanostructured material in which the hard phase is in the minority and hydrophilic, water penetrates the structure very rapidly and plasticizes the hydrophilic domains. The hydrophobic elastomer, which absorbs on average 1% to 2% by weight of water, does not confine the water between the two surfaces to be bonded but transports it towards the hydrophilic domains which act like sponges. The nanostructuring persists despite plasticization of said domains, which maintains the viscoelastic properties of the material and thus retains its adhesive properties.

As will be explained below, the Applicant has shown that in a moist medium, its block copolymers endow adhesive formulations with very rapid adhesion, with high water permeability and with ease of use without post-curing.

In a first aspect, the invention concerns an adhesive composition for moist medium adhesion comprising, as the binder, a block copolymer having at least one rigid hydrophilic block (B) constituting the minor phase dispersed in the form of nanodomains and at least one hydrophobic block (A) with an elastomeric nature having a water absorption capacity of less than 20%, constituting the continuous major phase.

The copolymers of the invention have the following general formula:

[(A)x−(B)]n

in which x is between 1 and 8, n is a whole number in the range 1 to 3, A and B respectively represent from 50% to 99%, preferably from 65% to 95% by weight, and from 1% to 50%, preferably from 10% to 35% by weight of the total copolymer weight. The number average molar mass (Mn) of the copolymer is in the range from 5000 g/mol to 300000 g/mol with a polydispersity index in the range from 1.1 to 3.

In accordance with the invention, the adhesive force of the composition or formulation depends on the difference in water absorption capacity of each block, hereinafter designated w_{∞} and is estimated as indicated below and expressed as a %.

Method for Determining w_{∞}:

A thin layer of a dry sample of polymer is prepared then exposed to an atmosphere with 98% humidity, thermostatted at 22° C. The water absorption is then measured using the equation:

$$w ( t ) = \frac{m ( t ) - m °}{m °} \%$$

where m(t) designates the mass of the sample as a function of time, m° the initial moist mass and w_{∞} then corresponds to the plateau of w(t) reached after a long period.

In the case of polydimethylacrylamide, the water absorption is 113% while that for butyl polyacrylate is 1.6%. The diffusion of water through a material follows Fick's law:

For w<006 w_{∞},

$$\frac{w ( t )}{{w}_{∞}} = \frac{4}{e} * {( \frac{D * t}{π} )}^{1 / 2} ,$$

in which e designates the thickness of the sample. In the case of polydimethylacrylamide, for example, the diffusion D is 3.10⁻¹³.s⁻¹.

As claimed in the invention, the optimum in terms of adhesive force is reached with a ratio w_{∞} (B)/w_{∞} (A) of more than 1, preferably more than 10 and more precisely more than 40.

In accordance with the preferred mode of the invention, w_{∞} (A) is less than 5% and w_{∞} (B)/w_{∞} (A) is more than 20.

A has a glass transition temperature (Tg(A)) of less than 30° C. and is thus qualified as a block with an elastomeric nature. In contrast, B must have a Tg (Tg(B)) of more than 50° C. Preferably, Tg(A) is in the range from −120° C. to 0° C., Tg(B) is in the range from 20° C. to 160° C.

The copolymers of the invention can be obtained using conventional polymerization techniques carried out in an organic or aqueous solution, by emulsion polymerization or by bulk polymerization, as described in the International Journal of Adhesion & Adhesives 22, 37-40 (2002). They are preferably prepared by controlled radical polymerization (CRP) in solution or in bulk.

Other techniques are possible but are more sensitive to the reactive functions of the monomers such as amides (for example by anionic polymerization). The advantage of CRP is that it is highly tolerant as regards the choice of monomers and for this reason, the Applicant used this synthesis technique.

The preferred preparation mode of the invention is that described in French patent application FR-99 01998 of 18 Feb. 1999, publication number FR-A-2 789 991.

A is obtained by polymerizing at least one monomer selected from the group containing hydrophobic monomers such as acrylic esters, for example butyl acrylate or hexyl acrylate, conjugated dienes. Preferably, butyl acrylate is used.

B is obtained by polymerizing at least one monomer selected from the group containing hydrophilic monomers such as acrylic acid, methacrylic acid, acrylamide, dimethylacrylamide, vinylpyrrolidone. It is preferably dimethylacrylamide. Monovalent salts of acrylic or methacrylic acids such as the sodium or lithium salts are also included in these hydrophilic monomers.

A and B can respectively contain residues of hydrophilic and hydrophobic monomers provided that the ratio of their water absorption capacity remains within the range defined above.

Nanostructuring of the copolymer is the resultant of a set of parameters such as chemical nature and block length. Whatever the choice of A and B, it is vital that the nanostructuring is preserved. Considerations that allow the nanostructuring to be preserved are known to the skilled person and reference can be made to the reference text: G Holden et al, in “Thermoplastic elastomers”, 2^{nd} edition, Carl Hanser Verlag, Munich, Vienna, N.Y., 1996.

The compositions of the invention also contain all of the additives necessary to shape them as well as the additives necessitated by the applications. The skilled person knows how to select these additives and how to define the processing conditions as a function of the envisaged application.

In all of its implementations, the compositions of the invention do not require post curing after processing.

The adhesive formulations of the invention can be used in a moist medium, in particular as regards any adhesion to the skin for a number of medical applications (patches, prostheses . . . ) or paramedical applications (dressings) which must adhere even after passage through water, as well as in the field of labeling.

The following examples illustrate the invention without limiting its scope.

### EXAMPLES

Examples 1 to 9 are in accordance with the invention. Examples 10 and 11 are comparative.

PDMA-b-PAbu (poly(N, N-dimethylacrylamide)-block-n-butyl polyacrylate) copolymers were synthesized by starting from the PDMA block. PDMA was synthesized by controlled radical polymerization (CRP). The control agent was N-tert-butyl-1-diethylphosphono-2,2-dimethylpropyl nitroxide, hereinafter designated SG1.

### Purification of Reagents

Poly(N,N-dimethylacrylamide) was stored in a desiccator under vacuum or under nitrogen. The n-butyl acrylate was cryodistilled over calcium hydride and stored in a refrigerator in a burette with ground graduations under nitrogen.

Azo-bis-isobutyronitrile (AIBN) was re-crystallized from ether and vacuum dried prior to being stored in a refrigerator in a flask under nitrogen.

The SG1 (83% and 90%) was used as supplied.

The solvents for precipitation were of analytical grade and used without purification.

### Synthesis

The calculated masses (see below) for the macro-initiator (PDMA) and SG1_{exc} were introduced into a Schlenk tube provided with a bar magnet. The Schlenk tube was connected to the burette, which latter was placed under a slight over-pressure of nitrogen to add the desired volume of monomer. The reaction medium was homogenized well and underwent 3-4 vacuum (Schlenk tube immersed in liquid nitrogen)/nitrogen cycles to remove all traces of oxygen. The Schlenk tube was then immersed in an oil bath at 110° C.

The polymer obtained was dissolved in THF, precipitated from a methanol/water=2/1 mixture at 0° C. and then filtered through a frit (no. 4, at 0° C.). In the case in which a portion of the copolymer passed through the frit, the filtrate was centrifuged (10000 rpm, 10 min, ambient temperature).

The residue was oven dried at 40° C. under vacuum (not exceeding 50° C.!) for 2-3 days.

### Calculation of Masses

The calculated masses of PDMA and SG1 depended on the total mass in and on the envisaged number average molar mass Mn of the copolymer, as well as on the conversion. These three parameters had to be selected initially (see paragraph above). The chains were “protected” by the SG1. However, to keep the polymolecularity indices as low as possible, addition of an excess of SG1 was recommended. Then the mass M_{b11} of the first block and the mass m_{SG1} of SG1 were calculated as follows:

Mass of first block:

### Universal Formula:

$${m}_{b 11} = \frac{\overline{{M}_{nb 11}} \times {V}_{mono\_b12 + solv} \times {d}_{mono\_b12} \times conv .}{{M}_{nb 12 , viseo} \times {n}_{radicaux}}$$

in which:
m_{b11}=mass of block 1
Mn=number average molar mass
Vₘₒₙₒ_{_{—}}_{b12+solv}=block monomer 2 volume plus solvent volume
dₘₒₙₒ_{_{—}}_{b12}=density of block monomer 2
n_{radicaux}=number of radicals per initiator molecule
conv.=degree of conversion (n_{monomer t?0}/n_{monomer t=0})

Here:

$${M}_{PDMA} = \frac{\overline{{M}_{nPDMA}} \times {m}_{Abu} \times conv .}{{M}_{nAbuviseo}}$$

Mass of excess SG1:

Preferably, the concentration ratios [SG1_{exc}]/[(macro-)initiator]=0.2-1.0 are used.

Here:

m_{SG1}=293×(“ratio”)×m_{PDMA}/ M̅_̅{̅e̅P̅D̅M̅A̅}̅

The characteristics of the products obtained are summarized in Table 1.

Application: practical results.

A/Preparation of adhesive formulations

The adhesives were prepared as follows:
450 μl of a 15% solution of copolymer in dioxane were deposited on an anodized aluminum plate (2.5×2.5×0.1 cm). A tackifying or plasticizing resin could be added to the solution in proportions that are known to the skilled person.

The solvent was evaporated off over two days at ambient temperature (in a Petri dish) before drying it completely in a vacuum oven at 50° C. for two additional days. The films obtained were 90±10 μm thick.

B/Description of the method for measuring adhesion

The tack properties of the copolymers were measured on a dry and moist surface using the probe tack test. It consisted of:
a compression step: a probe was brought towards the adhesive until a nominal contact force F, is attained;
a relaxation step: the adhesive was allowed to relax for a contact time t_{c} at constant thickness;
a tensile step: the probe was withdrawn at a constant velocity v.

The experiments were carried out using a tensile machine sold by the company Zwick. A motor produced the vertical displacement of the probe. This latter was connected to a 100 N force sensor fixed to a crossbeam. The force sensor and a position sensor provided us with access to the force and position of the crossbeam at any time (t). The experiments were carried out at ambient temperature, F_{c}=35 N, t_{c}=100 s and v=10 mm/min.

The probe used was a cylinder with a planar surface (7 mm diameter) formed from stainless steel. For the moist surface measurements, the probe was immersed in a beaker filled with distilled water so that one drop covered its entire surface.

The tack curves represented only the portion relating to separation of the bond in the stress-strain coordinates. The stress a was obtained by normalizing the force using the contact area. This latter was determined by measuring the area of the trace left by the probe on the adhesive, once the experiment was finished. The strain d was that of the probe.

The tack energy G corresponds to the integral of the curve in stress-strain coordinates and represents the energy that has to be provided to break the adhesive bond.

1. Dry medium adhesion measurement
The results are shown in Table 1.
A comparison of the samples prepared at 1 with a homopolymeric polybutyl acrylate (PABu) shows the importance of nanostructuring in obtaining the property of adhesion. A comparison with the formulation starting from Kraton (SBS) type triblock copolymer shows that in a dry medium, the diblock copolymers prepared at 1 are less adhesive than a triblock formulation.

2. Moist medium adhesion measurements:
The results are shown in Table 1.

A comparison shows that the formulations based on copolymers 2 or 3 bonded better than the reference formulation based on SBS.

TABLE 1
Characteristics of tested products and results obtained:

| | ADHESION RESULTS | | | | Moist |
|---|---|---|---|---|---|
| | Mn | Poly- | Dry medium | | medium |
| Test | co- | dispersity | Modulus | | Modulus |
| no | polymer | index | (G/cm²) | Type | (G/cm²) |
| 1 | 83 000 | 1.20 | 120 | Cohesive | |
| 2 | 73 000 | 1.29 | 120 | Cohesive | 245 |
| 3 | 158 000 | 1.40 | 380 | Adhesive | 125 |
| 4 | 54 400 | | 100 | Cohesive | |
| 5 | 63 000 | | 100 | Cohesive | |
| 6 | 72 000 | | 110 | Cohesive | |
| 7 | 94 000 | | 180 | Cohesive | |
| 8 | 75 000 | | 120 | Cohesive | |
| 9 | 85 000 | | 135 | Cohesive | |
| 10: | | | 600 | Adhesive | 25 |
| SBS | | | | | |
| 11: | 65 000 | 1.2 | 40 | | |
| PABu | | | | | |

1.-18. (canceled)

19. A block copolymer having at least one rigid hydrophilic block (B) as a minor phase dispersed in the form of nanodomains, said hydrophilic block obtained by polymerizing a hydrophilic monomer which is acrylic acid, acrylamide or dimethylacrylamide, and at least one hydrophobic block (A) with an elastomeric nature as a major phase and having a water absorption capacity woo (A) of less than 20%, obtained by polymerizing butyl acrylate, said copolymer having the structure [(A)ₓ−(B)ₙ, in which x is in the range 1 to 8, n is a whole number 1 to 3, A and B respectively represent from 65% to 95% by weight, and from 5% to 35% by weight of the total copolymer weight.

20. The block copolymer as claimed in claim 19, wherein w∞ (A) is less than 10%.

21. The block copolymer as claimed in claim 19, wherein the water absorption capacities of A, w∞ (A), and of B, w∞ (B), are such that the ratio woo (B)/w∞ (A) is more than 1.

22. The block copolymer as claimed in claim 21, wherein w∞ (A) is less than 5 and w∞ (B)/w∞ (A) is more than 20.

23. The block copolymer as claimed in claim 19, wherein the number average molar mass (Mn) of the block copolymer is 5,000 g/mol to 300,000 g/μmol, with a polydispersity index of 1.1 to 3.

24. The block copolymer as claimed in claim 19, wherein B has a glass transition temperature (Tg(B)) of more than 50° C.

25. The block copolymer as claimed in claim 19, wherein said hydrophilic block A has a glass transition temperature (Tg(A)) of less than 30° C.

26. The block copolymer as claimed in claim 19, wherein B is obtained by polymerizing dimethylacrylamide.

27. An adhesive composition comprising a solution of a binder, or a binder in combination with a resin or plasticizer, wherein the binder is the block copolymer according to claim 19.

28. A method of attaching a medical patch, dressing or prosthesis to the skin of a host, comprising contacting with the skin a patch, prosthesis or dressing to which an adhesive according to claim 27 has been applied.
